(19) United States
(12) Patent Application Publication
(10) Pub. No.: US 2009/0226504 A1
(43) Pub. Date: Sep. 10, 2009

(54) Intravascular, Interstitial Or Intraorgan Medical Access Device, And Manufacturing Method Thereof, Involving Nitric Oxide

(75) Inventor: Tor Peters, Helsingborg (SE)

Correspondence Address: INSKEEP INTELLECTUAL PROPERTY GROUP, INC, 2281 W. 190TH STREET, SUITE 200, TORRANCE, CA, 90504, US

(73) Assignee: NOLabs AB, Helsingborg (SE)

(21) Appl. No.: 11/909,155

(22) PCT Filed: Feb. 13, 2006
(86) PCT No.: PCT/EP2006/050896

§ 371 Date: Jun. 23, 2008

Related U.S. Application Data
(60) Provisional application No. 60/666,502, filed on Mar. 30, 2005.
(60) Provisional application No. 60/711,006, filed on Aug. 24, 2005.

(30) Foreign Application Priority Data
Mar. 24, 2005 (EP) 05006489.8
Aug. 23, 2005 (EP) 05018269.0

(51) Int. Cl. A61L 31/16; A61K 33/00

(52) U.S. Cl. 424/426; 424/718

(57) ABSTRACT

A intravascular, interstitial or intraorgan medical device, and a manufacturing process of said medical device, is provided that allows for prevention of infection and obtainment of anti-thrombotic effect. The medical device comprises a nitric oxide (NO) eluting polymer arranged adjacent mammal tissue, such that a therapeutic dose of nitric oxide is eluted from said nitric oxide eluting polymer to said mammal tissue. The nitric oxide (NO) eluting polymer is integrated with a carrier material, such that said carrier material, in use, regulates and controls the elution of said therapeutic dosage of nitric oxide (NO). Furthermore, a manufacturing method for said device is disclosed.

### RELATED APPLICATIONS

This application claims priority to International Patent Application No. PCT/EP2006/050896, International Filing Date 13 Feb. 2006, entitled “Intravascular, Interstitial Or Intraorgan Medical Access Device, And Manufacturing Method Thereof, Involving Nitric Oxide,” which claims priority from European Patent Application No. 05006489.8 filed 24 Mar. 2005 entitled “Intravascular, Interstitial Or Intraorgan Medical Access Device, And Manufacturing Method Thereof,” U.S. Provisional Application No. 60/666,502 filed Mar. 30, 2005 entitled “Intravascular, Interstitial Or Intraorgan Medical Access Device, And Manufacturing Method Thereof,” European Patent Application No. 05018269.0 filed 23 Aug. 2005 entitled “Device, System, And Method Comprising Microencapsulated Liquid For Release Of Nitric Oxide From A Polymer,” and U.S. Provisional Application Ser. No. 60/711,006 filed Aug. 24, 2005 entitled “Device, System, And Method Comprising Microencapsulated Liquid For Release Of Nitric Oxide From A Polymer,” all of which are hereby incorporated by reference.

### FIELD OF THE INVENTION

This invention pertains in general to the field of a medical device, involving the use of nitric oxide (NO). More particularly the invention relates to an intravascular, interstitial or intraorgan medical access device, and a process for manufacturing of said device, involving the use of nitric oxide (NO).

### BACKGROUND OF THE INVENTION

In the field of medicine a lot of medical devices are inserted, implanted, or attached to the body of a mammal, such as a human. These devices are intended to fulfill different medical purposes, such as closing a wound or operation wound, draining different kinds of body fluids from for example intraorgan, such as the pleura, urinary bladder, inner ear, or the vasculatory system, etc., for instance by the aid of catheters, and injecting medicaments, drugs, saline etc. Some medical devices are simply in contact with the mammal body, such as colostomy bags etc, or intended for supervision of blood pressure, blood gases etc. When these medical devices are inserted, implanted, or attached to the mammal body, the risk of infection is seriously increased.

Catheters are flexible rubber, or plastic, tubes that are inserted into different parts of the body, to provide a channel for fluid passage or another medical device. A catheter may for example remove waste fluids from the body after transurethral resection, or surgical operations in the lung.

A urinary catheter, such as a Foley catheter or balloon catheter, is inserted into the urinary bladder to drain urine. Because it can be left in place in the urinary-bladder for a period of time, it is also called an indwelling catheter. It is held in place with a balloon in the end, which is filled with sterile water, or air, in order to hold the catheter in place. Since the catheter is in place for a period of time, a number of side effects may occur, such as infections from bacteria, fewer, urosepsis etc. The bacteria present in, and in the vicinity of, the catheter may also be stone forming bacteria, which may result in blockage of the catheter. These disorders are today treated with antibiotics, but it is today common knowledge that treatment with antibiotics may result in development of bacteriological resistance against antibiotics, which may lead to severe complications in case of infections.

An intravenous catheter is inserted in a venous blood vessel to facilitate repeated injections, infusions, transfusions and blood samplings, and include central vein catheters (CVC), peripheral vein catheters (PVC), and subcutaneous vein ports (SVP). Also this type of catheter may cause infection, which infection today is treated with antibiotics entailing the side effects according to above. However, it is important to suppress this infections in order not to lead to local and systemic infectious complications, including local site infection, septic thrombophlebitis, endocarditis, and other metastatic infections, e.g., lung abscess, brain abscess, osteomyelitis, and endophthalmitis.

Another problem that may arise in the intravenous catheters according to the prior art is blockage of the catheters, due to coagulation of blood present in the catheters. Therefore, the catheters according to prior art have to be flushed with saline before and after each injection, infusion, transfusion and blood sampling, to ensure faultless infusion or injection.

In respect of closing wounds or operation wounds, sutures and staples are the most commonly used medical devices, in respect of both internal and external wounds. These staples and sutures are to keep the margins of the skin or tissue closed. These staples or sutures must be removed within 14 days from application. Otherwise they may cause complications, in form of infections, which infections today are treated with antibiotics entailing the side effects according to above.

Nitric oxide (NO) is a highly reactive molecule that is involved in many cell functions. In fact, nitric oxide plays a crucial role in the immune system and is utilized as an effector molecule by macrophages to protect itself against a number of pathogens, such as fungi, viruses, bacteria etc., and general microbial invasion. This improvement of healing is partly caused by NO inhibiting the activation or aggregation of blood platelets, and also by NO causing a reduction of inflammatory processes at the site of an implant.

NO is also known to have an anti-pathogenic, especially an anti-viral, effect, and furthermore NO has an anti-cancerous effect, as it is cytotoxic and cytostatic in therapeutic concentrations, i.e. it has among other effects tumoricidal and bacteriocidal effects. NO has for instance cytotoxic effects on human haematological malignant cells from patients with leukaemia or lymphoma, whereby NO may be used as a chemotherapeutic agent for treating such haematological disorders, even when the cells have become resistant to conventional anti-cancer drugs. This anti-pathogenic and anti-tumour effect of NO is taken advantage of by the present invention, without having adverse effects.

However, due to the short half-life of NO, it has hitherto been very hard to treat viral, bacteria, virus, fungi or yeast infections with NO. This is because NO is actually toxic in high concentrations and has negative effects when applied in too large amounts to the body.

NO is actually also a vasodilator, and too large amounts of NO introduced into the body will cause a complete collapse of the circulatory system. On the other hand, NO has a very short half-life of fractions of a second up to a few seconds, once it is released. Hence, administration limitations due to short half-life and toxicity of NO have been limiting factors in the use of NO in the field of anti-pathogenic and anti-cancerous treatment so far.

In recent years research has been directed to polymers with the capability of releasing nitrogen oxide when getting in contact with water. Such polymers are for example polyalkyleneimines, such as L-PEI (Linear PolyEthylenelmine) and B-PEI (Branched PolyEthylenelmine), which polymers have the advantage of being biocompatible with natural products, after the release of nitrogen oxide.

Other example for NO eluting polymers are given in U.S. Pat. No. 5,770,645, wherein polymers derivatized with at least one —NOₓ group per 1200 atomic mass unit of the polymer are disclosed, X being one or two. One example is an S-nitrosylated polymer and is prepared by reacting a polythiolated polymer with a nitrosylating agent under conditions suitable for nitrosylating free thiol groups.

Akron University has developed NO-eluting L-PEI molecule that can be nano-spun onto the surface of medical devices to be permanently implanted in the body, such as implanted grafts, showing significant improvement of the healing process and reduced inflammation when implanting such devices. According to U.S. Pat. No. 6,737,447, a coating for medical devices provides nitric oxide delivery using nanofibers of linear poly(ethylenimine)-diazeniumdiolate. Linear poly(ethylenimine) diazeniumdiolate releases nitric oxide (NO) in a controlled manner to tissues and organs to aid the healing process and to prevent injury to tissues at risk of injury.

However, the meaning of “controlled” in the context of U.S. Pat. No. 6,737,447 is only directed to the fact that nitric oxide is eluted from the coating during a period of time. Therefore, the interpretation of “controlled” in respect of U.S. Pat. No. 6,737,447 is different from the meaning of “regulating” in the present invention. “Regulate”, according to the present invention is intended to be interpreted as the possibility to vary the elution of nitric oxide to thereby achieve different elution profiles.

Electrospun nano-fibers of linear poly(ethylenimine) diazeniumdiolate deliver therapeutic levels of NO to the tissues surrounding a medical device while minimizing the alteration of the properties of the device. A nanofiber coating, because of the small size and large surface area per unit mass of the nanofibers, provides a much larger surface area per unit mass while minimizing changes in other properties of the device.

US 2001/041184 discloses biocompatible metallic medical devices capable of sustained nitric oxide release. These metallic devices are silanized with compounds having integral nucleophile residues, such as amine-functionalized silanes. This procedure is provided with a step of preliminary bind a nucleophile residue to a metallic surface, which also renders the coating according to US 2001/041184 restricted to metallic devices. Furthermore, the elution of nitric oxide from the device according to US 2001/041184 is not regulated in any way.

US 2004/0131753 discloses a coating for medical devices, which coating provides NO delivery by using nanofibers of L-PEI. It is unclear how the elution of nitric oxide according to US 2004/0131753 is initiated. As a matter of fact, US 2004/0131753 points out, and stresses, that the coating is insoluble in water. This may be interpreted as the release of NO is initiated by something else than water. Furthermore, the elution of nitric oxide from the coating according to US 2004/0131753 is not regulated in any way.

WO 02/17880 describes hydrogels releasing or producing nitric oxide. The hydrogels may be manufactured in the form of films, coatings, or micro-particles, which may be applied on medical devices, such as stents, vascular grafts, and catheters. Thus, the elution of nitric oxide from the hydrogel according to WO 02/17880 is not regulated in any way. US 2004/0259840 discloses nitric oxide releasing lipid molecules. These lipids may be integrated in a polymeric matrix. It is the lipid molecules that elute nitric oxide and not the polymer matrix. Furthermore, the elution of nitric oxide from the lipids according to US 2004/0259840 is not regulated in any way.

U.S. Pat. No. 6,261,594 discloses a chitosan based nitric oxide donor composition, comprising a modified chitosan polymer, for wound dressings. The elution of nitric oxide from the composition according to U.S. Pat. No. 6,261,594 is not regulated in any way.

However, the disclosure is both silent concerning an improvement of present technology in respect of medical devices for preventing infection and obtaining antithrombotic effect by the use of NO. Hence, an improved, or more advantageous, intravascular, interstitial or intraorgan medical access device, and a manufacturing process therefore, for preventing infection, which device presents an wound healing promoting and anti-infectious, anti-microbial, anti-inflammatory, anti-thrombotic, and/or anti-viral effect, would be advantageous.

### OBJECTS AND SUMMARY OF THE INVENTION

Accordingly, the present invention preferably seeks to mitigate, alleviate or eliminate one or more of the above-identified deficiencies in the art and disadvantages singly or in any combination and solves, among others, at least some of the problems mentioned above, by providing a medical device, for preventing infection and obtaining anti-thrombotic effect, a manufacturing method for the latter and a use of nitric oxide according to the appended patent claims.

According to one aspect of the invention, a medical device is provided that allows for prevention of infection and obtainment of anti-thrombotic effect. The device comprises a nitric oxide (NO) eluting polymer adjacent to an area of mammal tissue or body fluid, such that a therapeutic dose of nitric oxide is eluted from said nitric oxide eluting polymer to said area.

According to another aspect of the invention, a manufacturing process for such a medical device is provided, wherein the process is a process for forming a device that allows for prevention of infection and obtainment of anti-thrombotic effect. The process comprises selecting a plurality of nitric oxide eluting polymeric particles, such as nano fibres, fibres, nano particles, or microspheres, and deploying said nitric oxide eluting particles in, or on, said medical device.

The present invention has at least the advantage over the prior art that it provides a medical device with target exposure to NO, whereby prevention of infection and obtainment of anti-thrombotic effect, simultaneously as an anti-viral, an anti-inflammatory, and an anti-microbial therapy, are achievable.

### BRIEF DESCRIPTION OF THE DRAWINGS

These and other aspects, features and advantages of which the invention is capable of will be apparent and elucidated from the following description of embodiments of the present invention, reference being made to the accompanying drawings, in which

FIG. 1 is a schematic illustration of a catheter according to an embodiment of the invention, and

FIG. 2 is an illustration of two different elution profiles for two different mixtures of nitric oxide eluting polymer and carrier material.

### DETAILED DESCRIPTION OF THE INVENTION

The following description focuses on embodiments of the present invention applicable to a intravascular, interstitial or intraorgan medical device, which device allows for target exposure to NO, whereby prevention of infection and obtainment of anti-thrombotic effect is achieved, simultaneously as an anti-viral, an anti-inflammatory, and an anti-microbial therapy may be provided.

With regard to nitric oxide (nitrogen monoxide, NO), its physiological and pharmacological roles have attracted much attention and thus have been studied. NO is synthesized from arginine as the substrate by nitric oxide synthase (NOS). NOS is classified into a constitutive enzyme, cNOS, which is present even in the normal state of a living body and an inducible enzyme, iNOS, which is produced in a large amount in response to a certain stimulus. It is known that, as compared with the concentration of NO produced by cNOS, the concentration of NO produced by iNOS is 2 to 3 orders higher, and that iNOS produces an extremely large amount of NO.

In the case of the generation of a large amount of NO as in the case of the production by iNOS, it is known that NO reacts with active oxygen to attack exogenous microorganisms and cancer cells, but also to cause inflammation and tissue injury. On the other hand, in the case of the generation of a small amount of NO as in the case of the production by cNOS, it is considered that NO takes charge of various protective actions for a living body through cyclic GMP (cGMP), such as vasodilator action, improvement of the blood circulation, anti-platelet-aggregating action, anti-bacterial action, anti-viral action, anti-inflammatory action, anticancer action, acceleration of the absorption at the digestive tract, renal function regulation, neurotransmitting action, erection (reproduction), learning, appetite, and the like. Heretofore, inhibitors of the enzymatic activity of NOS have been examined for the purpose of preventing inflammation and tissue injury, which are considered to be attributable to NO generated in a large amount in a living body. However, the promotion of the enzymatic activity (or expressed amount) of NOS (in particular, cNOS) has not been examined for the purpose of exhibiting various protective actions for a living body by promoting the enzymatic activity of NOS and producing NO appropriately.

In recent years research has been directed to polymers with the capability of releasing nitrogen oxide when getting in contact with water. Such polymers are for example polyalkyleneimines, such as L-PEI (Linear PolyEthylenelmine) and B-PEI (Branched PolyEthylenelmine), which polymers have the advantage of being biocompatible. Another advantage is that NO is released without any-secondary products that could lead to undesired side effects.

The polymers according to the present invention may be manufactured by electro spinning, gas spinning, air spinning, wet spinning, dry spinning, melt spinning, and gel spinning. Electro spinning is a process by which a suspended polymer is charged. At a characteristic voltage a fine jet of polymer releases from the surface in response to the tensile forces generated by interaction by an applied electric field with the electrical charge carried by the jet. This process produces a bundle of polymer fibres, such as nano-fibres. This jet of polymer fibres may be directed to a surface to be treated.

Furthermore, U.S. Pat. No. 6,382,526, U.S. Pat. No. 6,520,425, and U.S. Pat. No. 6,695,992 disclose processes and apparatuses for the production of such polymeric fibres. These techniques are generally based on gas stream spinning, also known within the fiber forming industry as air spinning, of liquids and/or solutions capable of forming fibers.

Other example for NO eluting polymers are given in U.S. Pat. No. 5,770,645, wherein polymers derivatized with at least one —NOX group per 1200 atomic mass unit of the polymer are disclosed, X being one or two. One example is an S-nitrosylated polymer and is prepared by reacting a polythiolated polymer with a nitrosylating agent under conditions suitable for nitrosylating free thiol groups.

Akron University has developed NO-eluting L-PEI molecule that can be nano-spun onto the surface of permanently implanted medical devices, such as implanted grafts, showing significant improvement of the healing process and reduced inflammation when implanting such devices. According to U.S. Pat. No. 6,737,447, a coating for medical devices provides nitric oxide delivery using nanofibers of linear poly(ethylenimine)-diazeniumdiolate. Linear poly(ethylenimine) diazeniumdiolate releases nitric oxide (NO) in a controlled manner.

However, the meaning of “controlled” in the context of U.S. Pat. No. 6,737,447 is only directed to the fact that nitric oxide is eluted from the coating during a period of time, i.e. that the nitric oxide not is eluted all in once. Therefore, the interpretation of “controlled” in respect of U.S. Pat. No. 6,737,447 is different from the meaning of “regulating” in the present invention. “Regulate or control”, according to the present invention is intended to be interpreted as the possibility to vary the elution of nitric oxide to thereby achieve different elution profiles.

A polymer comprising an O-nitrosylated group is also a possible nitric oxide eluting polymer. Thus, in one embodiment of the present invention, the nitric oxide eluting polymer comprises diazeniumdiolate groups, S-nitrosylated and O-nitrosylated groups, or any combinations thereof.

In still another embodiment of the present invention said nitric oxide eluting polymer is a poly(alkyleneimine) diazeniumdiolate, such as L-PEI-NO (linear poly (ethyleneimine) diazeniumdiolate), where said nitric oxide eluting polymer is loaded with nitric oxide through the diazeniumdiolate groups and arranged to release nitric oxide at a treatment site.

Some other examples of a suitable nitric oxide eluting polymer are selected from the group comprising amino cellulose, amino dextrans, chitosan, aminated chitosan, polyethyleneimine, PEI-cellulose, polypropyleneimine, polybutyleneimine, polyurethane, poly(butanediol spermate), poly(iminocarbonate), polypeptide, Carboxy Methyl Cellulose (CMC), polystyrene, poly(vinyl chloride), and polydimethylsiloxane, or any combinations of these, and these mentioned polymers grafted to an inert backbone, such as a polysaccharide backbone or cellulosic backbone.

In still another embodiment of the present invention the nitric oxide eluting polymer may be a O-derivatized NONOate. This kind of polymer often needs an enzymatic reaction to release nitric oxide.

Other ways of describing polymers, which may be suitable as nitric oxide eluting polymer, is polymers comprising secondary amine groups (═N—H), such as L-PEI, or have a secondary amine (═N—H) as a pendant, such as aminocellulose.

The nitric oxide eluting polymer may comprise a secondary amine, either in the backbone or as a pendant, as described previously. This will make a good nitric oxide eluting polymer. The secondary amine should have a strong negative charge to be easy to load with nitric oxide. If there is a ligand close to the secondary amine, such as on a neighbour atom, such as a carbon atom, to the nitrogen atom, with higher electronegativity than nitrogen (N), it is very difficult to load the polymer with nitric oxide. On the other hand, if there is a electropositive ligand close to the secondary amine, such as on a neighbour atom, such as a carbon atom, to the nitrogen atom, the electronegativity of the amine will increase and thereby increase the possibility to load the nitric oxide elution polymer with nitric oxide.

In an embodiment of the present invention the nitric oxide polymer may be stabilized with a salt. Since the nitric oxide eluting group, such as a diazeniumdiolate group, usually is negative, a positive counter ion, such as a cation, may be used to stabilize the nitric oxide eluting group. This cation may for example be selected from the group comprising any cation from group 1 or group 2 in the periodic table, such as Na+, K+, Li+, Be²+, Ca²+, Mg²+, Ba²+, and/or Sr²+. Different salts of the same nitric oxide eluting polymer have different properties. In this way a suitable salt (or cation) may be selected for different purposes. Examples of cationic stabilized polymers are L-PEI-NO-Na, i.e. L-PEI diazeniumdiolate stabilized with sodium, and L-PEI-NO-Ca, i.e. L-PEI diazeniumdiolate stabilized with calcium.

Another embodiment of the present invention comprises mixing the nitric oxide eluting polymer, or a mixture of the nitric oxide eluting polymer and a carrier material, with an absorbent agent. This embodiment provides the advantage of an accelerated elution of nitric oxide since the polymer, or polymer mixture, via the absorbent agent, may take up the activating fluid, such as water or body fluid, much faster. In one example 80% (w/w) absorbent agent is mixed with the nitric oxide eluting polymer, or mixture of nitric oxide eluting polymer and carrier material, and in another embodiment 10 to 50% (w/w) absorbent agent is mixed with the nitric oxide eluting polymer, or mixture of nitric oxide eluting polymer and carrier material.

Since the elution of nitric oxide is activated by a proton donor, such as water, it may be an advantage to keep the nitric oxide eluting polymer, or mixture of nitric oxide eluting polymer and carrier material, in contact with said proton donor. If an indication requires an elution of nitric oxide during a prolonged period of time, a system is advantageous, which presents the possibility to keep the proton donor in contact with the nitric oxide eluting polymer, or mixture of nitric oxide eluting polymer and carrier material. Therefore, in still another embodiment of the present invention, the elution of nitric oxide may be regulated by adding an absorbent agent. The absorbent agent absorbs the proton donor, such as water, and keeps the proton donor in close contact with the nitric oxide eluting polymer during prolonged periods of time. Said absorbent agent may be selected from the group comprising polyacrylates, polyethylene oxide, carboxymethylcellulose, and microcrystalline cellulose, cotton, and starch. This absorbent agent may also be used as a filling agent. In this case said filling agent may give the nitric oxide eluting polymer, or mixture of said nitric oxide eluting polymer and a carrier material, a desired texture.

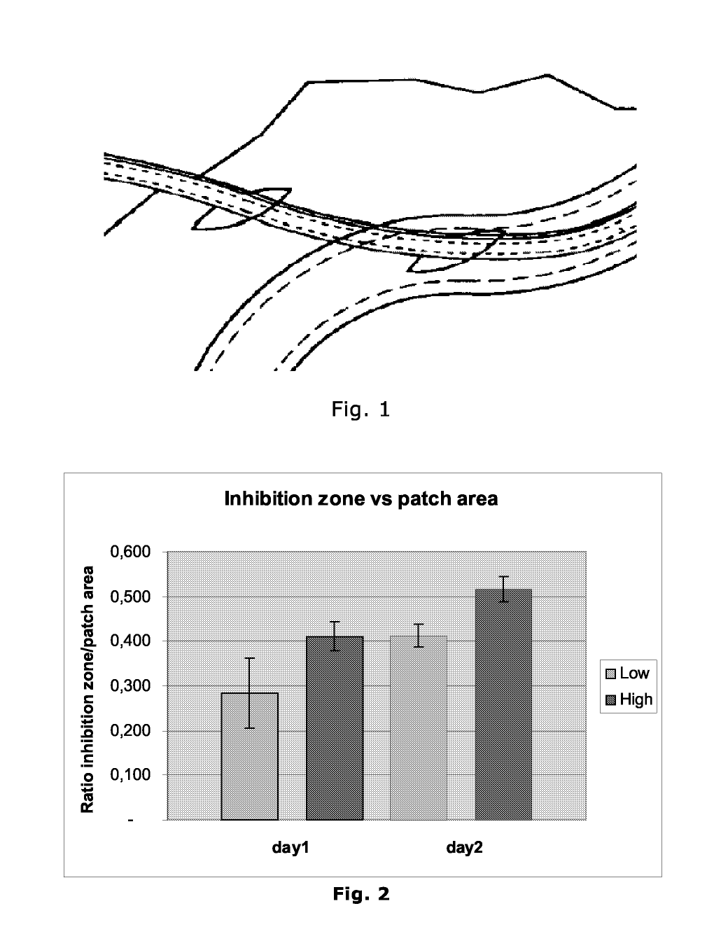

In one embodiment the device is in form of a catheter, according to FIG. 1, which catheter is suitable to be used for draining different kinds of body fluids from for example pleura, urinary bladder, blood system, ear, etc., by the aid of catheters, and injecting medicaments, drugs, saline etc.

The core material of the catheter according to the present invention may be any suitable material according to the prior art, such as polyethylene, polypropylene, polyacrylonitrile, polyurethane, polyvinylacetates, polylacticacids, starch, cellulose, polyhydroxyalkanoates, polyesters, polycaprolactone, polyvinylalcohol, polystyrene, polyethers, polycarbonates, polyamides, poly(acrylic acid), Carboxy Methyl Cellulose (CMC), protein based polymers, gelatine, biodegradable polymers, cotton, latex, silicone, polytetrafluoroethene, polyvinylchloride, polycarbonate, Acrylonitrile Butadiene Styrene (ABS), polyacrylate, polyolefins, polystyrene, rubbers, and/or any combinations of these.

The surface of said core material is then covered with the NO eluting polymers according to the present invention. This is accomplished by electro spinning, air spinning, gas spinning, wet spinning, dry spinning, melt spinning, or gel spinning of said NO eluting polymer onto said core material. In another embodiment of the present invention the NO eluting polymer according to the invention is integrated in the core material. This embodiment has the advantage of easier presentation of NO eluting polymer on the surface of the catheter facing the body fluid, such as blood, to thereby obtaining an anti-thrombotic effect on this side of the catheter.

This may be accomplished by integrating fibres, nano-particles or micro-spheres of the NO-eluting polymer according to the present invention in the core material prior to the moulding of said catheter.

These fibres, nano-particles, or micro-spheres, may be formed from the NO-eluting polymers comprised in the present invention, for example polyalkyleneimines, such as L-PEI (Linear PolyEthylenelmine) and B-PEI (Branched PolyEthylenelmine), which polymers have the advantage of being biocompatible, after the release of nitrogen oxide.

They may also be encapsulated in any suitable material, such as polyethylene, polypropylene, polyacrylonitrile, polyurethane, polyvinylacetates, polylacticacids, starch, cellulose, polyhydroxyalkanoates, polyesters, polycaprolactone, polyvinylalcohol, polystyrene, polyethers, polycarbonates, polyamides, polyolefins, poly(acrylic acid), Carboxy Methyl Cellulose (CMC), protein based polymers, gelatine, biodegradable polymers, cotton, and latex, or any combinations of these. This encapsulation is performed if, by any reason, the elution of NO needs to be regulated in respect of time.

In the context of the present invention the term “encapsulating” is intended to be interpreted as fixating the nitric oxide eluting polymer in a three dimensional matrix such as a foam, a film, a nonwoven mat of nanofibers or fibers, other materials with the capability to fixate the NO eluting polymer, or enclosing the nitric oxide eluting polymer in any suitable material.

Three important factors in controlling and regulating the elution of nitric oxide from a nitric oxide eluting polymer are how quickly a proton donor, such as water or body fluid, comes in contact with the nitric oxide releasing polymer, such as a diazoliumdiolate group, the acidity of the environment surrounding the nitric oxide eluting polymer, and the temperature of the environment surrounding the nitric oxide releasing polymer (higher temperature promotes elution of nitric oxide).

In one embodiment of the present invention a nitric oxide eluting polymer, such as L-PEI-NO, is mixed with a carrier polymer to slow down or prolong the elution of nitric oxide. Also, in another embodiment, the nitric oxide eluting polymer may be mixed with more than one carrier polymer, whereby be elution or release may be tailor made to fit specific needs. Such a need may for example be a low elution during a first period of time, when the environment of the nitric oxide eluting polymer is hydrophobic, and a faster elution during a second period of time, when the environment of the nitric oxide eluting polymer has been altered to be more hydrophilic. This may for example be accomplished by using biodegradable polymers, whereby a low elution during a first period of time is obtained, after which, when the hydrophobic polymer has been dissolved, the hydrophilic polymer provides a higher elution of nitric oxide. Thus, a more hydrophobic carrier polymer will give a slower elution of nitric oxide, since the activating proton donor, such as water or body fluid, will penetrate the carrier polymer slower. On the other hand, a hydrophilic polymer acts the opposite way. One example of an hydrophilic polymer is polyethylene oxide, and one example of an hydrophobic polymer is polystyrene. These carrier polymers may be mixed with the nitric oxide eluting polymer and then electrospun to suitable fibers. The skilled person in the art knows which other polymers may be used for similar purposes. FIG. 2 illustrates two elution profiles (NO concentration vs. time) for two different polymer mixtures; a nitric oxide eluting polymer mixed with a hydrophilic carrier polymer in an acidic environment (A), and a nitric oxide eluting polymer mixed with a hydrophobic carrier polymer in a neutral environment (B).

In one embodiment this carrier polymer is substituted by another material with hydrophobic or hydrophilic properties. Therefore, the term “carrier material” in the present context should be interpreted to include carrier polymers and other materials with hydrophilic or hydrophobic properties.

In another embodiment of the present invention the elution of nitric oxide from a nitric oxide eluting polymer, such as L-PEI-NO, is influenced by the presence of protons. This means that a more acidic environment provides a quicker elution of nitric oxide. By activating the nitric oxide eluting polymer, or mixture of nitric oxide eluting polymer and carrier material, with an acidic fluid, such as an ascorbic acid solution, the elution of nitric oxide may be accelerated.

The carrier polymers and carrier materials mentioned above may affect other characteristics than the regulation of nitric oxide elution. An example of such characteristic is mechanical strength.

In respect of the carrier polymers or carrier materials, the NO-eluting polymer may be integrated in, spun together with, or spun on top of, any of these materials in all of the embodiments of the present invention. This spinning includes electro spinning, air spinning, dry spinning, wet spinning, melt spinning, and gel spinning. In this way, one may manufacture fibers of a polymer mixture, comprising a nitric oxide eluting polymer and a carrier polymer, or a carrier material, with predefined nitric oxide eluting characteristics. These characteristics may be tailor made for different elution profiles in different applications.

In still another embodiment the nitric oxide eluting polymer, such as powder, nano-particles or micro-spheres, can be incorporated in foam. The foam may have an open cell structure, which facilitates the transport of the proton donor to the nitric oxide eluting polymer. The foam can be of any suitable polymer such as polyethylene, polypropylene, polyacrylonitrile, polyurethane, polyvinylacetates, polylacticacids, starch, cellulose, polyhydroxyalkanoates, polyesters, polycaprolactone, polyvinylalcohol, polystyrene, polyethers, polycarbonates, polyamides, poly(acrylic acid), Carboxy Methyl Cellulose (CMC), protein based polymers, gelatine, biodegradable polymers, cotton, polyolefins, and latex, or any combinations of these, or latex.

The device is applied on the intended area, such as urethra, bloodstream, pleura, pharynx, trachea, etc.

When the device has been applied, an elution of NO is initiated when the device, including the NO eluting polymer according to the present invention, gets in contact with the moisture or water in the adjacent tissue of the mammal body.

A therapeutic effect of the application area is obtained, as the NO eluting polymer elutes NO on the application area, whereby an anti-microbial, anti-inflammatory, anti-thrombotic or anti-viral effect of the tissue of interest is achieved.

The increased blood perfusion and vasodilatation may, in another embodiment of the present invention, result in an improved effect when combined with other active components. Thus, the synergistic effect from NO and other active components is within the scope of the present invention.

The Seldinger technique is a method for percutaneous puncture and catheterization of the arterial system, also called percutaneous vascular catherization. It is named after Sven-Ivar Seldinger, a Swedish radiologist. The method is based on, following local anaesthesia and a small skin incision, that the artery is punctured using a thin-walled needle, e.g. with 1.0 to 1.2 mm outer diameter with or without a central mandril. The needle is advanced into the artery at an angle of approximately 45. After removing the mandril, the needle is pulled back till a pulsating back flow is seen. Then a guide wire with a flexible tip, (usually a J-guide wire) is advanced into the vessel. Under manual compression the needle is withdrawn and a catheter is advanced over the guide wire into the artery and positioned at the desired location. The guide wire is then pulled back and the catheter is checked for back flow and carefully rinsed with saline. The Seldinger technique comprises the following steps: 1) puncturing of a vessel, such as an artery with a thin walled percutaneous entry needle, 2) removal of mandril, passing a guide wire through the lumen of the entry needle, advancing a portion of the guide wire length into the vessel 3) withdrawing of the needle, 4) optionally enlarging the puncture site with a scalpel, 5) advancing of a catheter over the guide wire into the vessel, for instance with a twisting motion, and 6) after the catheter is in position, removing the guide wire, now the catheter is ready for use.

The same technique can be used also for catheterization of other tubular structures such as the bile ducts, the collecting system of the kidney as well as for abscess drainages etc.

According to certain embodiments of the access device of the present invention, catheters used for the Seldinger technique incorporate NO eluting polymers as described herein.

In embodiments of the present invention the device may be selected from the group consisting of venflones; catheters, such as urinary catheters, central vein catheters (CVC), peripheral vein catheters (PVC), and subcutaneous vein ports (SVP); drainage tubes, such as tubes for pleura drainage and other wound drainages; articles intended for supervision of pressure and/or blood gases; and intravenous dressings.

When the device is in form of a urinary catheter, said urinary catheter is provided with anti-microbial, anti-inflammatory, anti-thrombotic and anti-viral effect. Thereby, the urinary catheter may by in place for a long period of time, while still conquering the majority of the side effects according to the prior art, such as infections from bacteria, fewer, urosepsis, and/or bacteriological stone formation. Furthermore, there is hence no need for treatment with antibiotics.

When the device is in form of a venflone, a central vein catheter, a peripheral vein catheter, and a subcutaneous vein port, said devices are provided with anti-microbial, anti-inflammatory, anti-thrombotic and anti-viral effect. Thereby, said devices may be in place for a long period of time, while still conquering the majority of side effects according to the prior art, such as infections from bacteria, and blockage of the devices, due to coagulation of blood present in the catheters. Therefore, there is no need for flushing the devices according to the invention prior to and after each injection, infusion, transfusion and blood sampling, to ensure faultless infusion or injection.

When the device is in form of a drainage tube, said drainage tube is provided with anti-microbial, anti-inflammatory, anti-thrombotic and anti-viral effect. Thereby, said devices may be in place for a long period of time, while still conquering the majority of side effects according to the prior art, such as infections from bacteria, and blockage of the drainage tube, due to coagulation of blood present in the drainage tube.

When the device is in form of an article for the supervision of blood pressure or blood gases, said device is provided with anti-microbial, anti-inflammatory, antithrombotic and anti-viral effect. Thereby, said device may be in place for a long period of time, while still conquering the majority of side effects according to the prior art, such as infections from bacteria, and blockage of the devices, due to coagulation of blood present in the catheters.

In another embodiment the device is in form of sutures or staples. The sutures and staples according to the invention are provided with anti-microbial, anti-inflammatory, and anti-viral effect. Thereby, said sutures and staples may be in place for a long period of time, while still conquering the majority of side effects according to the prior art, such as infections from bacteria. Therefore, the staples or sutures according to the present invention need not be removed within 14 days from application, which is the case with the sutures and staples according to the prior art. Since the sutures and staples according to the invention provides an anti-inflammatory effect, the risk of need for treatment with antibiotics is significantly reduced.

When the device is in form of a intravenous dressing, the area surrounding an intravenous catheter is provided with anti-microbial, anti-inflammatory, anti-thrombotic and anti-viral effect. This embodiment has the advantage of protecting an area which otherwise is exposed to a high possibility of getting in contact with infectious material.

The NO-eluting polymer may be integrated in, spun together with, or spun on top of, any of these devices in all of the embodiments of the present invention.

Preferably the aforementioned embodiments employ L-PEI material loaded with NO. Activation on NO release will be achieved when the devices according to all the embodiments of the present invention get in contact with the moisture and/or water of the adjacent tissue of the mammal.

In another embodiment the fibres, nano-particles, or micro-spheres, may be integrated in a soluble film that disintegrates on the inside of the devices according to the present invention, in order to elute NO at the area of interest when the soluble film gets in contact with the moisture or water in the adjacent tissue of the mammal.

In another embodiment of the present invention the device only allows NO-elution in one direction. In this kind of embodiment one side of the device according to the invention is non-permeable to NO. This may be accomplished by applying a material on one side of the device according to the invention that has low permeability, substantially no permeability, or no permeability to nitric oxide. Such materials may be chosen from the group comprising common plastics, such as fluoropolymers, polyethylene, polypropylene, polyacrylonitrile, polyurethane, polyvinylacetates, polylacticacids, starch, cellulose, polyhydroxyalkanoates, polyesters, polycaprolactone, polyvinylalcohol, polystyrene, polyethers, polycarbonates, polyamides, polyolefins, poly(acrylic acid), Carboxy Methyl Cellulose (CMC), protein based polymers, gelatine, biodegradable polymers, cotton, and latex, or any combinations of these. This embodiment is also easy to manufacture as the NO eluting polymer, e.g. L-PEI nano fibres may be electro or gas-jet spun onto the surface of the device according to the invention of e.g. the mentioned plastics, latex, or cotton. This may protect the NO eluting polymer during packaging, transport and prior to use from external influences, being e.g. mechanical (abrasion of the polymer), chemical (moisture deactivating the device prior to use) etc.

In yet another embodiment of the present invention the NO-eluting device is acting as a booster for medications and pharmaceuticals. This embodiment presents a device with the advantage of combining two treatments, of significant value, in one treatment.

Hence, such devices may achieve a synergetic effect, when NO is eluted from the devices. NO has a vasodilatory effect. Vasodilated tissue is more susceptible to certain medications and pharmaceuticals, and thus more easily treated by the medical preparations and still NO has the anti-inflammatory, anti-bacterial, anti-thrombotic, and anti-viral effect. Hence, an unexpected surprisingly effective treatment is provided.

Catheters are normally manufactured by extrusion. When manufacturing catheters and venflones according to the present invention, the NO eluting polymer may be integrated in the polymer blend that will be extruded. This manufacturing process provides catheters and venflones with the ability to elute NO to the fluid, or body fluid, passing through said catheters/venflones.

In another manufacturing process according to the present invention the catheters and venflones are manufactured in a two step process. In the first step the catheters/venflones are extruded. In the second step the catheters/venflones are covered on the in- and outside with NO eluting polymer by electro-spinning, air spinning, gas spinning, wet spinning, dry spinning, melt spinning, or gel spinning.

The device elutes nitric oxide (NO) from said eluting polymer in a therapeutic dose, such as between 0.001 to 5000 ppm, such as 0.01 to 3000 ppm, such as 0.1 to 1000 ppm, such as 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90 91, 92, 93, 94, 95, 96, 97, 98, 99, or 100 ppm. The concentration may vary widely depending on where the concentration is measured. If the concentration is measured close to the actual NO eluting polymer the concentration may be as high as thousands of ppm, while the concentration inside the tissue in this case often is considerably lower, such as between 1 to 1000 ppm.

The NO-eluting polymers in the devices according to the present invention may be combined with silver, such as hydroactivated silver. The integration of silver in the devices according to the present invention gives the therapeutic treatment an extra boost. Preferably the silver is releasable from the devices in the form of silver ions. The integration of silver in the device may present several advantages. One example of such an advantage is that the silver may keep the device in itself free from bacteria or viruses, while the nitric oxide eluting polymer elutes the therapeutic dosage of nitric oxide to the target site.

The device may be manufactured by, for example electro spinning of L-PEI or other polymers comprising L-PEI or being arranged in combination with L-PEI. L-PEI is the charged at a characteristic voltage, and a fine jet of L-PEI releases as a bundle of L-PEI polymer fibres. This jet of polymer fibres may be directed to a surface to be treated. The surface to be treated may for example be any suitable material. The electro spun fibres of L-PEI then attach on said material and form a coating/layer of L-PEI on the device according to the invention.

It is of course possible to electro spin the other NO-eluting polymers, according to above, on the device according to the invention while still being inside the scope of the present invention.

In one embodiment the NO-eluting polymers according to the present invention are electro spun in such way that pure NO-eluting polymer fibres may be obtained. It is also within the scope of the present invention to electro spin a NO-eluting polymer together with other suitable polymer/polymers.

Gas stream spinning, air spinning, wet spinning, dry spinning, melt spinning, and gel spinning, of said NO-eluting polymers onto the device is also within the scope of the present invention.

The manufacturing process according to the present invention presents the advantages of large contact surface of the NO-eluting polymer fibres with the area to be treated, effective use of NO-eluting polymer, and a cost effective way of producing the device.

The invention may be implemented in any suitable form. The elements and components of the embodiments according to the invention may be physically, functionally, and logically implemented in any suitable way. Indeed, the functionality may be implemented in a single unit, in a plurality of units, or as part of other functional units.

Although the present invention has been described above with reference to specific embodiments, it is not intended to be limited to the specific form set forth herein. Rather, the invention is limited only by the accompanying claims and, other embodiments than the specific above are equally possible within the scope of these appended claims.

In the claims, the term “comprises/comprising” does not exclude the presence of other elements or steps. Furthermore, although individually listed, a plurality of means, elements or method steps may be implemented. Additionally, although individual features may be included in different claims, these may possibly advantageously be combined, and the inclusion in different claims does not imply that a combination of features is not feasible and/or advantageous. In addition, singular references do not exclude a plurality. The terms “a”, “an”, “first”, “second” etc do not preclude a plurality. Reference signs in the claims are provided merely as a clarifying example and shall not be construed as limiting the scope of the claims in any way.

Although the invention has been described in terms of particular embodiments and applications, one of ordinary skill in the art, in light of this teaching, can generate additional embodiments and modifications without departing from the spirit of or exceeding the scope of the claimed invention. Accordingly, it is to be understood that the drawings and descriptions herein are proffered by way of example to facilitate comprehension of the invention and should not be construed to limit the scope thereof.

What is claimed is:

1. An intravascular-, interstitial- or intraorgan-medical access device that allows for prevention of an infection and/or obtainment of an anti-thrombotic effect,
wherein
said access device comprises a nitric oxide (NO) eluting polymer configured to elute a therapeutic dosage of nitric oxide (NO), and
wherein said access device is configured to expose an adjacent area of mammal tissue to said nitric oxide when said polymer, in use, elutes nitric oxide (NO),
characterized in that
said nitric oxide (NO) eluting polymer is integrated with a carrier material, such that said carrier material, in use, regulates and controls the elution of said therapeutic dosage of nitric oxide (NO) from said nitric oxide (NO) eluting polymer,
wherein said adjacent area of mammal tissue is at a site entering a mammal body comprising said mammal tissue,
such that said intravascular, interstitial or intraorgan medical access device in use provides prevention of said infection and/or obtainment of said anti-thrombotic effect at said site entering said mammal body.

2. Medical access device according to claim 1, wherein said medical access device comprises a core material which surface is covered with said nitric oxide (NO) eluting polymer at a position of said medical access device that in use of said medical access device is to be positioned at said site entering said mammal body.

3. Medical access device according to claim 1, wherein said medical device comprises a core material, and wherein said nitric oxide (NO) eluting polymer is integrated in said core material, such that nitric oxide (NO) eluting polymer is present on an internal surface of the medical access device.

4. Medical access device according to claim 1, wherein said medical access device is an indwelling catheter.

5. Medical access device according to claim 1, wherein said intravascular, interstitial or intraorgan medical access device is a body fluid draining catheter to be used for draining body fluids.

6. Medical access device according to claim 5, wherein said body fluid draining catheter further allows for injection of fluid into said mammal body.

7. Medical access device according to claim 1, wherein one side of the medical device is substantially non-permeable to nitric oxide (NO) such that said elution of nitric oxide (NO) from said medical access device in use substantially is directed towards one direction of said medical access device.

8. Medical access device according to claim 1, wherein said nitric oxide (NO) eluting polymer comprises diazeniumdiolate groups, S-nitrosylated groups, and O-nitrosylated groups, or any combination these.

9. Medical access device according to claim 1, wherein said nitric oxide (NO) eluting polymer is L-PEI (linear polyethyleneimine), loaded with nitric oxide (NO) through said diazeniumdiolate groups, S-nitrosylated groups, or O-nitrosylated groups, or any combination of these, arranged for release of the nitric oxide (NO) to said adjacent area of mammal tissue.

10. Medical access device according to claim 1, wherein said nitric oxide (NO) eluting polymer is selected from the group comprising amino cellulose, amino dextrans, chitosan, aminated chitosan, polyethyleneimine, PEI-cellulose, polypropyleneimine, polybutyleneimine, polyurethane, poly(butanediol spermate), poly(iminocarbonate), polypeptide, Carboxy Methyl Cellulose (CMC), polystyrene, poly(vinyl chloride), and polydimethylsiloxane, or any combinations of these, and these mentioned polymers grafted to an inert backbone, such as a polysaccharide backbone or cellulosic backbone.

11. Medical access device according to claim 1, selected from the group consisting of venflones; urinary catheters, central vein catheters (CVC), peripheral vein catheters (PVC), and subcutaneous vein ports (SVP); drainage tubes, including tubes for pleura drainage and other wound or organ drainages; articles intended for supervision of pressure and/or blood gases; gaskets for colostomy bags; tubes for pharynx and trachea; and intravenous dressings.

12. Medical access device according to claim 11, including said nitric oxide (NO) eluting polymer mixed with a core material, said core material comprising polyethylene, polypropylene, polyacrylonitrile, polyurethane, polyvinylacetates, polylacticacids, starch, cellulose, polyhydroxyalkanoates, polyesters, polycaprolactone, polyvinylalcohol, polystyrene, polyethers, polycarbonates, polyamides, poly(acrylic acid), Carboxy Methyl Cellulose (CMC), protein based polymers, gelatine, biodegradable polymers, cotton, latex, silicone, polytetrafluoroethene, polyvinylchloride, polycarbonate, Acrylonitrile Butadiene Styrene (ABS), polyacrylate, polyolefins, polystyrene, rubbers, and/or any combinations of these.

13. Medical access device according to claim 1, wherein said medical access device is partly disintegrable when subjected to moisture or water.

14. Medical access device according to claim 1, wherein said medical access device comprises silver, configured for therapeutic treatment of said mammal tissue.

15. Medical access device according to claim 1, wherein said polymer is comprised in the medical access device in form of a powder, fibers, nano-particles or micro-spheres.

16. Medical access device according to claim 15, wherein said powder, nano-particles, or micro-spheres, are integrated with, preferably encapsulated in, a material, selected from the group consisting of polyethylene, polypropylene, polyacrylonitrile, polyurethane, polyvinylacetates, polylacticacids, starch, cellulose, polyhydroxyalkanoates, polyesters, polycaprolactone, polyvinylalcohol, polystyrene, polyethers, polycarbonates, polyamides, polyolefins, poly(acrylic acid), Carboxy Methyl Cellulose (CMC), protein based polymers, gelatine, biodegradable polymers, cotton, and latex, or any combinations of these.

17. Medical access device according to claim 1, wherein said carrier material is selected from the group comprising polyethylene, polypropylene, polyacrylonitrile, polyurethane, polyvinylacetates, polylacticacids, starch, cellulose, polyhydroxyalkanoates, polyesters, polycaprolactone, polyvinylalcohol, polystyrene, polyethers, polycarbonates, polyamides, polyolefins, poly(acrylic acid), Carbonyl Methyl Cellulose (CMC), protein based polymers, gelatine, biodegradable polymers, cotton, and latex, or any combinations of these.

18. Medical access device according to claim 1, wherein said nitric oxide eluting polymer comprises a secondary amine in the backbone or a secondary amine as a pendant.

19. Medical access device according to claim 18, wherein a positive ligand is located on a neighbor atom to the secondary amine.

20. Medical access device according to claim 1, comprising an absorbent agent.

21. Medical access device according to claim 20, wherein said absorbent agent is selected from the group comprising polyacrylate, polyethylene oxide, Carboxy Methyl Cellulose (CMC), microcrystalline cellulose, cotton, or starch, or any combinations thereof.

22. Medical access device according to claim 1, comprising a cation, said cation stabilizing the nitric oxide eluting polymer.

23. Medical access device according to claim 22, wherein said cation is selected from the group comprising Na⁺, K⁺, Li⁺, Be²*, Ca²⁺, Mg²\Ba²⁺, and/or Sr²⁺, or any combinations thereof.

24. A manufacturing process for an intravascular, interstitial or intraorgan medical access device that allows for prevention of an infection and/or obtainment of an anti-thrombotic effect at an adjacent area of mammal tissue at a site entering a mammal body comprising said mammal tissue, according to claim 1, comprising;
selecting a nitric oxide (NO) eluting polymer configured to elute a therapeutic dosage of nitric oxide (NO) when used-for said prevention of infection and/or obtainment of said anti-thrombotic effect,
selecting a carrier material, which carrier material is configured to regulate and control the elution of said therapeutic dosage of nitric oxide (HO) from said nitric oxide (NO) eluting polymer,
incorporating the nitric oxide (NO) eluting polymer with said carrier material into an nitric oxide (NO) eluting material, such, that said carrier material, in use of said medical access device, regulates and controls the elution of said therapeutic dosage of nitric oxide (NO), and
deploying said nitric oxide eluting material into a suitable form, or as a coating onto a carrier, to form at least a part of said medical access device at a position of said medical access device that in use of said medical access device is to be positioned at said site entering said mammal body, such that said medical access device is configured to expose said site adjacent to said device in use thereof to said nitric oxide when said nitric oxide (NO) eluting polymer in use elutes nitric oxide (NO), whereby said intravascular, interstitial or intraorgan medical access device in use provides prevention of infection and/or obtainment of anti-thrombotic effect at said site entering said mammal body.

25. The manufacturing process according to claim 24,
wherein said deploying comprises electro spinning, air spinning, gas spinning, wet spinning, dry spinning, melt spinning, or gel spinning of said nitric oxide (NO) eluting polymer.

26. The manufacturing process according to claim 24, wherein said selecting said nitric oxide (NO) eluting polymer comprises selecting a plurality of nitric oxide {NO) eluting polymeric particles, preferably powder, nano fibres, nano particles or micro spheres.

27. The manufacturing process according to claim 24, wherein said incorporating-said nitric oxide (NO) eluting polymer with said carrier material comprises integrating said nitric oxide (NO) eluting polymer in said carrier material, spinning said nitric oxide (NO) eluting polymer together with said carrier material, or spinning said nitric oxide (HO) eluting polymer on top of said carrier material, in order to predefine nitric oxide eluting characteristics of said device.

28. The manufacturing process according to claim 24, further comprising integrating silver in said device.

29. Use of a nitric oxide (NO) eluting polymer for the manufacture of a intravascular, interstitial or intraorgan medical access device according to claim 1,
wherein
nitric oxide (NO) is loaded to said device in such a way that said device elutes nitric oxide (NO) to adjacent mammal tissue from said eluting polymer in a therapeutic dose at a position of said medical access device that in use of the medical access device is to be positioned at a site entering a mammal body comprising said mammal tissue.

30. Use according to claim 29, wherein said therapeutic dose is 0.001 to 5000 ppm, such as 0.01 to 3000 ppm, such as 0.1 to 1000 ppm, such as 1, 2, 3, 4, 5, 6, 1, 8, 9, 10, 11, 12, 13, 14, 15, 16, 17, 18, 19/20, 21, 22, 23, 24, 25, 26, 27, 2B, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90 91, 92, 93, 94, 95, 96, 97, 98, 99, or 100 ppm.

31. A therapeutic method for prevention of infection and/or obtainment of anti-thrombotic effect when using a intravascular, interstitial or intraorgan medical access device, comprising deploying a intravascular, interstitial or intraorgan medical access device according to claim 1 to a site entering a mammal body, and
exposing an adjacent area of’ mammal tissue at said site entering said mammal body to nitric oxide eluted from a polymer of said medical access device during use thereof.
